(19) United States
(12) Patent Application Publication
(10) Pub. No.: US 2006/0127374 A1
(43) Pub. Date: Jun. 15, 2006

(54) Method for the in vitro production of cartilage-like tissues

(76) Inventors: Hans J. Hauselmann, Zurich (CH); Martin Stoddart, Zurich (CH); Erik Hedbom, Zurich (CH)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 11/284,410

(22) Filed: Nov. 21, 2005

Related U.S. Application Data
(63) Continuation of application No. PCT/CH04/00312, filed on May 24, 2004.

(30) Foreign Application Priority Data
May 23, 2003 (WO) PCT/IB03/02153
Jul. 29, 2003 (WO) PCT/IB03/03481
Jul. 30, 2003 (WO) PCT/IB03/03455

(51) Int. Cl. A61K 35/32; C12N 5/08

(52) U.S. Cl. 424/93.7; 435/366

(57) ABSTRACT

A method for the in vitro production of cartilage-like tissue is disclosed. Said method comprises the cultivation of chondrogenic cells in presence of bivalent cation dependent constituents of the extracellular matrix (BCDM) and/or human serum wherein an inhibitory activity of cartilage maturation has been selectively removed from said human serum. The resulting cartilage-like tissue can be used for the repair of chondral and/or osteochondral defects in subjects or animals.

### RELATED APPLICATION DATA

This application is a continuation of International Application No. PCT/CH2004/000312 filed 24 May 2004 and claims priority to International Application Nos. PCT/IB03/02153, filed 23 May 2003; PCT/IB03/03481 filed 29 Jul. 2003 and PCT/IB03/03455 filed 30 Jul. 2003.

### TECHNICAL FIELD

The present invention relates to a method for the production of cartilage-like tissue in vitro and implants comprising the inventive cartilage-like tissue for the treatment of chondral and/or osteochondral defects in human beings and animals.

### BACKGROUND ART

Articular cartilage lesions which occur frequently as a result of trauma or degenerative disease do not heal naturally and often lead to progressive cartilage degeneration. This problem can be seen as a lack of capacity to mobilize chondrocytes required to regenerate the lost tissue and, hence, transplantation of chondrocytes is a potential therapy for localized cartilage defects. Different methods for seeding of cartilage cells into the defect area are possible. One technique, which has been given particularly much attention, is based on the injection of a chondrocyte suspension under a sutured periostal membrane covering the defect (Brittberg, et al., N. Engl. J. Med 331:889-895, 1994). Other methods employ chondrocytes together with different biomaterial carriers as grafts for the repair of cartilage defects (Cao Y., et al., J Biomater Sci Polym Ed 9:475-487, 1998). Furthermore, a variety of methods used for pre-culturing of cells in vitro have opened interesting possibilities to create viable and implantable constructs. These methods yield cartilage which differs greatly from natural cartilage.

There is therefore a need for improved in vitro methods allowing the production of cartilage-like tissue for implantation into chondral and/or osteochondral defects of human beings and animals.

### DISCLOSURE OF THE INVENTION

Hence, it is a general object of the present invention to provide a method for the production of cartilage-like tissue in vitro. The method comprises the cultivation of chondrogenic cells in presence of BCDM (bivalent cation dependent constituents of extracellular matrix) and/or human serum wherein an inhibitory activity of cartilage maturation and/or growth has been selectively removed from said human serum.

Said inhibitory activity in human serum is preferably a factor or factors inhibiting the growth and/or maturation of cartilage, more preferably a factor or factors inhibiting the production of cartilage matrix in the cultivated cells.

The factor or factors inhibiting cartilage maturation, preferably cartilage matrix production, have preferably a molecular weight of less than 100,000 Dalton, more preferably less than 75,000 Dalton, even more preferably less than 50,000 Dalton, most preferably less than 25,000 Dalton.

In a particular preferred embodiment, the factor or factors have a molecular weight of less than 10,000 Dalton, more preferably less than 8,000 Dalton.

In a preferred embodiment the chondrogenic cells are cultivated in a media further comprising a factor selected from the group consisting of chondroitin sulfate, preferably chondroitin-4-sulfate and/or chondroitin-6-sulfate, interleukins, in particular interleukin 4, lipids, glucosamines, heparin sulphate, TGFβ, IGF-1, preferably IGF-1 long R3, dexamethasone, FGF2 and PDGFPβ. These factors can be added to the culture medium either alone or in any combination.

In a further preferred embodiment said chondrogenic cells are chondrocytes. Preferably, said chondrocytes derive from chondrogenic cells which are seeded in a alginate gel and cultured for a time sufficient for differentiation into chondrocytes and biosynthesis and assembly of extracellular matrix.

In a preferred embodiment said chondrogenic cells are multiplied by anchorage dependent growth before seeded in the alginate gel. Said chondrogenic cells originate preferably from a tissue biopsy or from mesenchymal stem cells, more preferably from a human being.

In a preferred embodiment said chondrogenic cells are cultivated in a confined space which is preferably a chamber whose confinements are in part porous or emipermeable. Preferably said chamber is at least in part made of a bone and/or bone substitute material, in particular hydroxyapatite or tricalcium phosphate.

In a further preferred embodiment said BCDM are added to cells released from alginate before cultivation in a confined space, in particular after multiplication of said chondrogenic cells. Said bivalent cation dependent constituents of the extracellular matrix (BCDM) are preferably isolated from a chondrocyte culture, more preferably an alginate chondrocyte culture.

In another preferred embodiment the in vitro produced cartilage-like tissue or a compound formed of said cartilage-like tissue and bone or bone substitute is in a further step subjected to a mechanical treatment.

In a preferred embodiment said mechanical treatment forces have parallel and perpendicular components with respect to a surface of said cartilage-like tissue or said compound formed of said cartilage-like tissue and bone or bone substitute. Preferably, said cartilage-like tissue or the compound formed of said cartilage-like tissue and bone or bone substitute is subjected to mechanical stimulation by intermittent compression/ deformation cycles.

A further object of the present invention is directed to in vitro cartilage-like tissue obtainable or obtained by a method of the present invention or a compound formed of said cartilage-like tissue and bone or bone substitute.

Yet a further object of the present invention relates to an implant for repairing a chondral and/or osteochondral defect comprising an in vitro cartilage-like tissue obtainable by a method of the present invention or a compound formed of said cartilage-like tissue and bone or bone substitute.

Furthermore, the present invention relates to a method for the treatment of a chondral and/or osteochondral defect in a subject wherein an in vitro cartilage-like tissue of the present invention and/or an implant of the present invention is applied to/implanted into said chondral and/or osteochondral defect. Preferably, said subject is a human being and said in vitro cartilage-like tissue is autologous tissue.

In a further aspect the present invention relates to a method of joining several tissue patches for forming a substantially closed tissue surface. Said method comprises the steps of: providing tissue patches of substantially polygonal shape, in particular tissue patches comprising cartilage-like tissue obtained by a method of the present invention and arranging said polygonal tissue patches side by side in such a manner that they form a substantially closed tissue surface.

The tissue patches used are preferably part of tissue/bone composites.

A further object of the present invention is a substantially closed tissue surface obtainable by the method of the present invention.

Yet another object of the present invention is an implant comprising the tissue surface of the present invention. Preferably said implant is a cartilage-like/bone composite.

### BRIEF DESCRIPTION OF THE DRAWINGS

The invention will be better understood and objects other than those set forth above will become apparent when consideration is given to the following detailed description thereof. Such description makes reference to the annexed drawings, wherein:

FIG. 1 shows a flow diagram of the method for the production of in vitro cartilage-like tissue;

FIG. 2A shows the schematic phenotype of a chondrocyte in monolayer culture;

FIG. 2B shows the schematic phenotype of a monolayer chondrocyte directly after being placed into a 3 dimensional culture system;

FIG. 2C shows the schematic phenotype of a chondrocyte after a period of 3 dimensional culture, demonstrating an increase in extracellular matrix;

FIG. 3 shows the inhibitory effect of human serum on cartilage matrix synthesis with respect to incorporation of 35S over a 16 hour labelling period;

FIG. 4A shows an exemplary polygonal osteochondral plug;

FIG. 4B shows an exemplary disc shaped osteochondral plug;

FIG. 5 shows an exemplary polygonal tissue patch;

FIG. 6 shows a tissue surface formed by polygonal tissue patches of the present invention;

FIGS. 7A and B show exemplary tissue plugs with interlocking edges.

FIGS. 8A and B show exemplary essentially polygonal tissue patches comprising a curved circumference line and

### MODES FOR CARRYING OUT THE INVENTION

The method of the present invention is characterised in that chondrogenic cells are cultivated in presence of BCDM and/or modified human serum.

The term “modified human serum” describes a human serum from which an inhibitory activity, preferably an inhibitory factor or inhibitory factors of cartilage maturation, preferably cartilage matrix production, have been removed selectively. It is possible that the factor or the factors inhibiting the growth and/or maturation of cartilage have a toxic activity. The modified human serum can be produced e.g. by dialysis of serum or fractionation of serum, collecting the different serum fractions and testing their ability to stimulate de novo cartilage production and/or maturation in vitro. The serum fractions showing the desired effect are then pooled and used in a method of the present invention.

The BCDM can be isolated from chondrocytes cultured in alginate beads. An exemplary isolation method comprises the following steps:
a) solubilization of the alginate beads by a buffer containing citrate,
b) centrifugation of the solution to yield a cell containing pellet and a supernatant,
c) addition of a bivalent cation (e.g. Calcium chloride) to the supernatant to precipitate the alginate,
d) centrifugation of the solution to pellet the alginate and
e) collecting the supernatant containing the BCDM.

The cultured chondrocytes can stem from humans or any animal.

The chondroitin sulphate, preferably chondroitin-4-sulphate and chondroitin-6-sulphate, can stem from various sources and are commercially available. The terms interleukins, lipids, TGFβ, IGF-1, heparin sulphate, glucosamine, dexamethasone, FGF2 and PDGFββ as used herein comprise their functional analogous and fragments. These factors are known to a person skilled in the art and can be isolated from different sources or be produced by recombinant methods. The cultivation media that can be used in a method of the present invention can as well contain further factors which are usually used for the in vitro cultivation of cells. Such factors are known to a person skilled in the art and commercially available.

The mechanical treatment of the in vitro produced cartilage-like tissue leads to cartilage-like tissue with improved characteristics compared to untreated cartilage and is therefore especially suitable for implantation into a chondral and/or osteochondral defect in subjects.

For clinical applications the in vitro produced cartilage-like tissue can e.g. be used in combination with tissue/bone composites to form implants of variable forms. Suitable materials that can serve as tissue/bone composites are known to a person skilled in the art and include e.g., tricalcium phosphate. The tissue patches/plugs of the present invention can be coated with factors which, e.g., enhance bone ingrowth. Such factors are known to a person skilled in the art and comprise e.g., one morphogenic proteins.

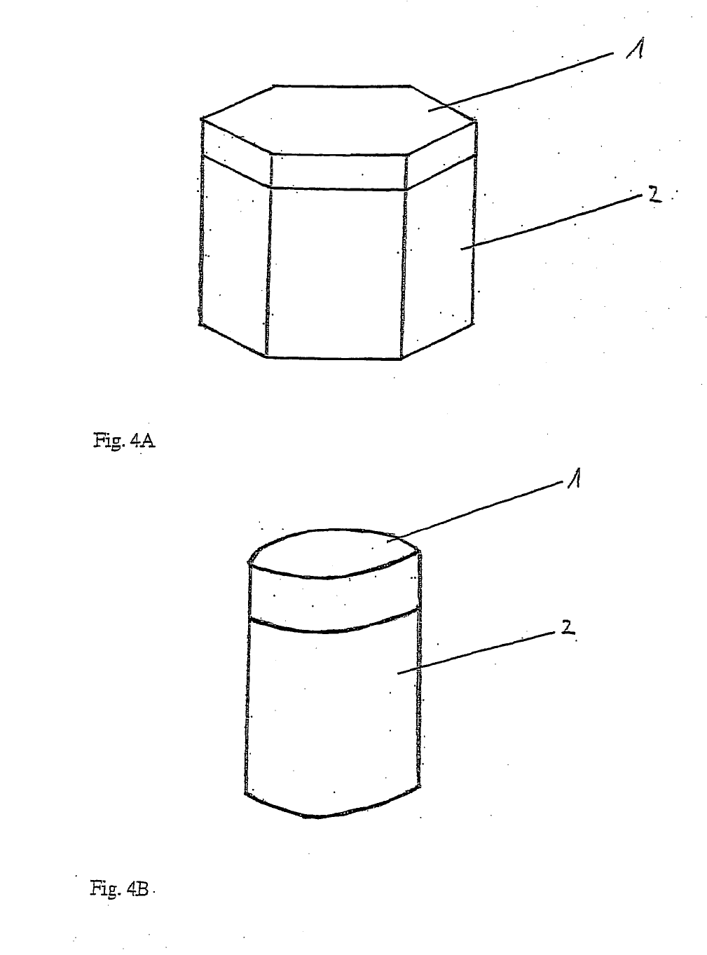

FIGS. 4A and 4B show exemplary osteochondral implants according to the present invention. The implant of FIG. 4A has the form of a polygonal plug and the implant of FIG. 4B is a disc shaped osteochondral plug. An osteochondral plug comprises a tissue part 1 and a part 2 made of a suitable tissue/bone composite.

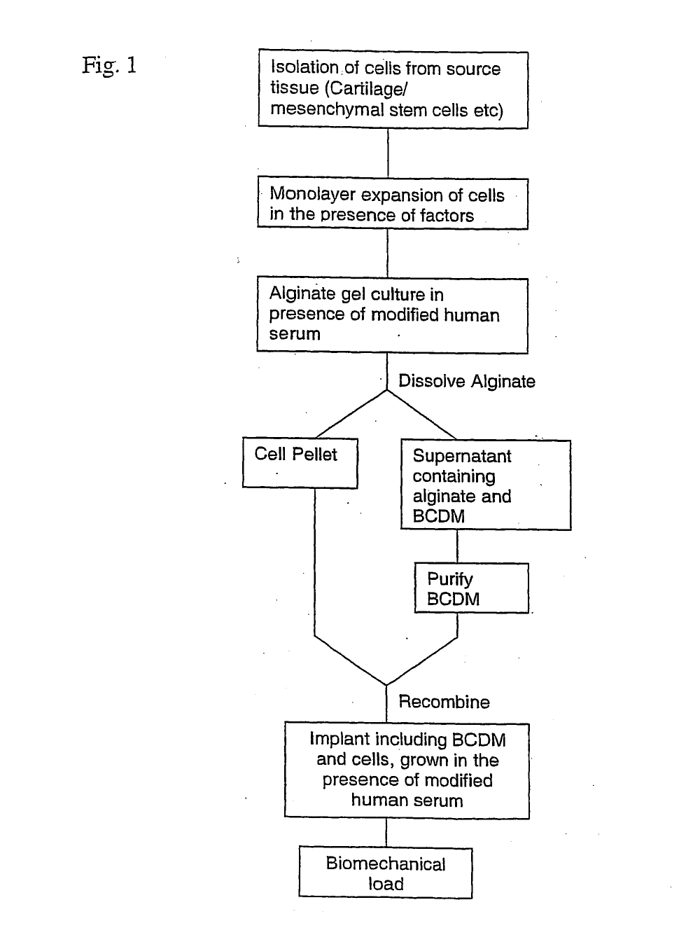

FIG. 1 shows a flow diagram of a preferred embodiment of the method of the present invention. In this preferred embodiment an alginate cell culture of chondrogenic cells, preferably chondrocytes, is cultured in presence of modified human serum. The resulting cells of this step are then in a further optional step cultivated in presence of BCDM.

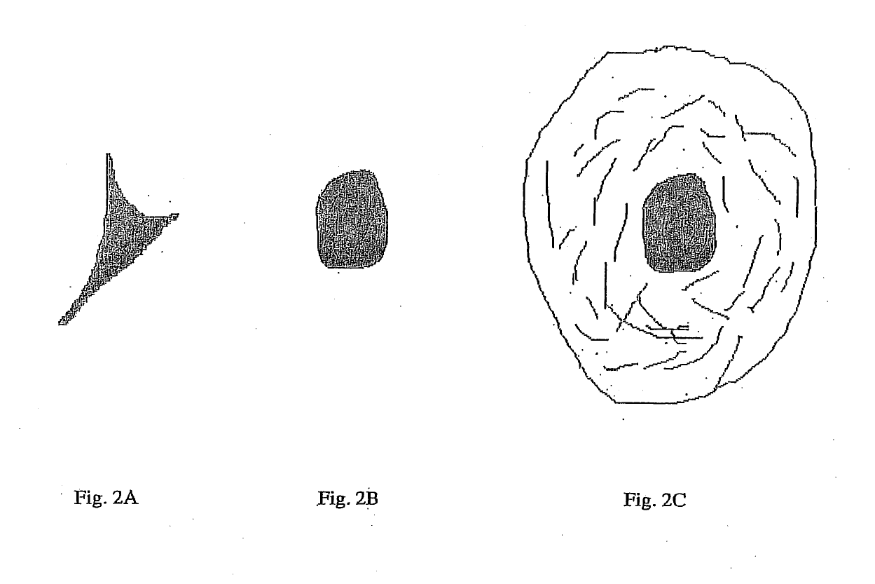

FIGS. 2A to 2C show schematics of phenotypes of chondrocytes cultivated under different conditions in vitro.

In FIG. 5 a hexagonal tissue patch is depicted. A tissue patch/plug of the present invention can have any essentially polygonal shape which can be joined to form a substantially closed surface. The term “substantially polygonal shape” as used herein encompasses shapes which comprise a curved circumference line. Two exemplary essentially polygonal patches with a curved circumference line are shown in FIGS. 8A and B.

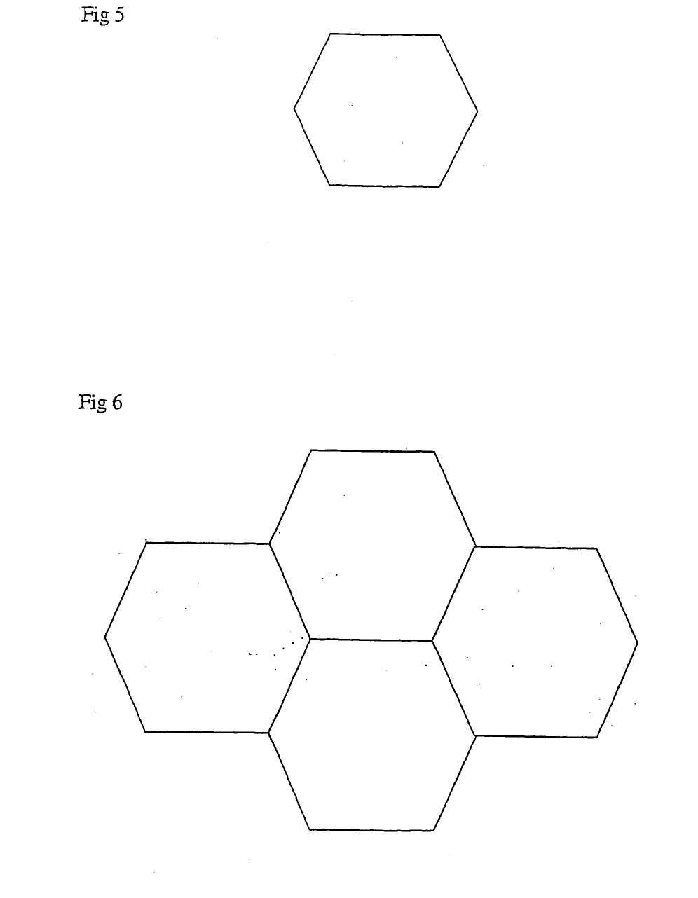

FIG. 6 shows a closed tissue surface formed by hexagonal tissue patches/plugs. Such an ex vivo formed tissue surface can then be implanted by a surgeon into a chondral and/or osteochondral lesion in a patient. The closed tissue surface of the present invention allows an easy implantation in a lesion of a patient compared to the implantation of single plugs and their assembly in the lesion of a patient. Depending on the size and shape of the patient's lesion a closed tissue surface can be assembled ex vivo and can easily be implanted in a single step in a patient.

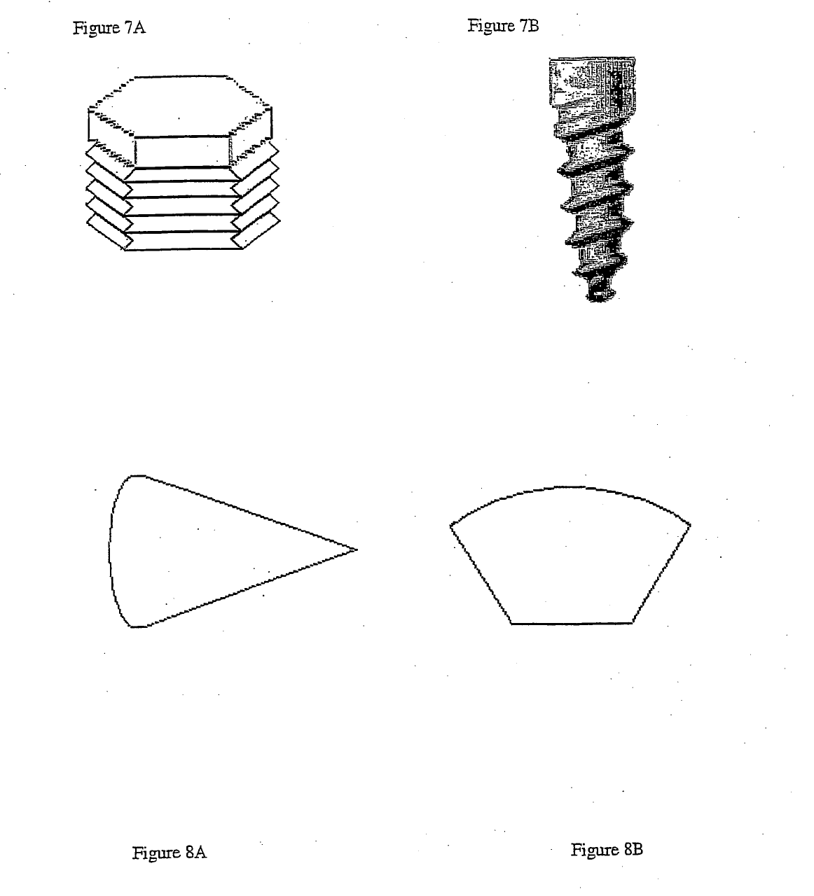

The tissue patches/plugs or an implant of the present invention can comprise interlocking edges in order to be stable when implanted in a lesion of a patient. Exemplary interlocking edges are e.g. roughened edges, spiral edges or incisions. Exemplary plugs/implants with exemplary interlocking edges are shown in FIGS. 7A and B.

The application is now further illustrated by means of examples.

### EXAMPLE I

### Preparation of BCDM

Feasibility studies were performed using chondrocytes from young (6-18 month old) freshly slaughtered bovine calves.

Culture Conditions

Full-thickness articular cartilage is dissected from the metacarpophalangeal joints. The cartilage slices are digested at 37° C. for 90 minutes with 0.4% Pronase (Calbiochem, La Jolla, Calif.) and then for 16 hours at 37° C. with 0.025% Collagenase P from Clostridium hystolyticum (Roche Diagnostics, Mannheim, Germany) in DMEMIF12 (Gibco Invitrogen, Basel, Switzerland) containing 5% fetal bovine serum. The digest is then filtered through a 40 μm cell strainer (Beckton Dickinson, Franklin Lakes, N.J.). The chondrocytes are then collected by centrifugation and resuspended in a 1.2% solution of sterile alginate (Kelton L V, Kelco, Chicago, USA) in 0.15 M NaCl at a density of 4×10⁶ cells/ml. The cell suspension is slowly expressed through a 22-gauge needle and dropped into a 102 mM calcium chloride solution. The newly formed beads are polymerised in this solution for 10 minutes and then washed twice in 0.15 M NaCl followed by two washes in DMEM/F12. The beads then are transferred to complete culture medium consisting of DMEM/F12, 50 μg/ml gentamicin, 10% FCS, an effective amount of additional growth factors and 25 μg/ml ascorbic acid (Gibco Invitrogen, Basel, Switzerland). The cultures are kept at 37° C. in a humidified atmosphere of 5% CO2 in air with the medium replaced by fresh medium once every two days.

After 14 days of culture, the medium is removed and the beads dissolved at 37° C. by incubation for 10 minutes in 55 mM sodium citrate, 0.15 M NaCl, pH 6.8 . The cell suspension is harvested by centrifugation. The pellet, containing the cells with their cell-associated matrix, is resuspended in 55 mM sodium citrate, 0.15 M NaCl, pH 6.8 and again incubated for 10 minutes at 37° C. After harvesting by centrifugation the pellet, containing the cells with their cell-associated matrix, then is then re-suspended 100% FCS, to produce the final cell slurry.

### EXAMPLE OF BCDM PREPARATION

Total supernatant from dissolving the beads is taken (65 ml) and dialyzed against 0.9% NaCl for 2 days.

To this CaCl2 is added (to a final concentration of 0.155 M) and the alginate is precipitated o/n with stirring followed by centrifugation at 4,000 rpm.

The supernatant is taken and passed through PM10 ultra filtration (10,000 mwt cut off) and the buffer is exchanged for 0.9% NaCl. The resultant solution is a viscous fluid, which is sterile filtered through a 0.22 μm filter. The BCDM can be mixed with the cells and their cell-associated matrix to enhance the cell slurry. Alternatively the viscous BCDM can be concentrated and/or dried using a speed vac (Savant Instruments, Forrningdale, N.Y.) prior to being mixed with the cell suspension.

### EXAMPLE II

### Demonstration of Human Serum's Inhibitory Effect on Human Chondrocytes

Feasibility studies were performed using chondrocytes from normal human cartilage, obtained during autopsy from the lateral femoral condyle according to strict ethical guidelines.

Culture Conditions

Full-thickness articular cartilage is dissected from the lateral femoral condyle from human cadavers. The cartilage slices are digested at 37° C. for 90 minutes with 0.4% Pronase (Calbiochem, La Jolla, Calif.) and then for 16 hours at 37° C. with 0.025% Collagenase P from Clostridium hystolyticum (Roche Diagnostics, Mannheim, Germany) in DMEM/F12 (Gibco Invitrogen, Basel, Switzerland) containing 5% human serum. The digest is then filtered through a 40 μm cell strainer (Beckton Dickinson, Franklin Lakes, N.J.) and the chondrocytes are harvested by centrifugation.

The chondrocytes are resuspended in complete medium consisting of DMEM/F12, 50 μg/ml gentamicin, 10% human serum, an effective amount of additional growth factors and 25 μg/ml ascorbic acid (Gibco Invitrogen, Basel, Switzerland) and plated on tissue culture plastic at a density of 1×104 cells/cm2 and the cultures are kept at 37° C. in a humidified atmosphere of 5% CO2 in air with the medium replaced by fresh medium once every two days. Once the cells reach confluence, approximately 16-18 days, they are washed three times with Hanks balanced salt solution (Sigma, St. Louis, USA) and harvested by incubation with Trypsin/EDTA in Hanks balanced salt solution (Gibco Invitrogen, Basel, Switzerland) at 37° C. for 10 min. The digestion is then stopped by the addition of DMEM/F12 containing 10% human serum and the cell number counted.

The cells are then collected by centrifugation and resuspended in a 1.2% solution of sterile alginate (Kelton L V, Kelco, Chicago, USA or NovaMatrix FMC, BioPolymer, Norway) in 0.15 M NaCl at a density of 4×106 cells/ml. The cell suspension is slowly expressed through a 22-gauge needle and dropped into a 102 mM calcium chloride solution. The newly formed beads are polymerised in this solution for 10 minutes and then washed twice in 0.15 M NaCl followed by two washes in DMEM/F12. The beads then are transferred to complete culture medium consisting of DMEM/F12, 50 μg/ml gentamicin, 25 μg/ml ascorbic acid (Gibco Invitrogen, Basel, Switzerland) and various concentrations of human and fetal calf serum as detailed in the results section. The cultures are kept at 37° C. in a humidified atmosphere of 5% CO2 in air with the medium replaced by fresh medium once every two days.

Characterization of Matrix Biosynthesis After 14 Days of Treatment in Alginate Beads

Assessment of Biosynthetic Activity by 35S Protein Labelling

After 14 days of treatment 3 beads are taken and incubated for 16 hours with 300 μl complete medium containing 50 μCi/ml 35S. The beads are papain digested overnight and then extracted with 8M guanidine hydrochloride. The resultant solution is fractionated over a PD10 column and the radiation contained in each fraction quantified by liquid scintillation counting. A portion of each sample is used to quantify the DNA content and all data is normalized to DNA content.

Results

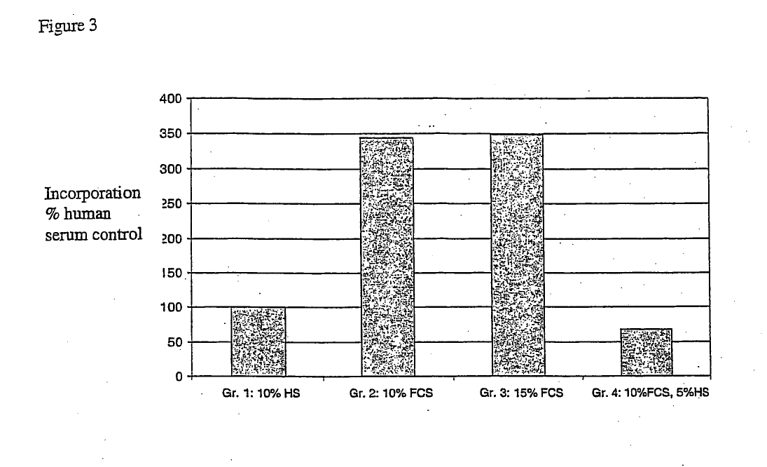

Human chondrocytes were cultured in alginate gel and treated with various serum concentrations for 14 days. Ten percent Human serum (HS) was used as a control. Biosynthetic activity was analyzed as described above. FCS at either 10% or 15% raised synthetic activity by approximately 350% of the control. Adding 5% HS to 10% FCS dramatically reduced this increase to levels comparable with 10% HS alone (see FIG. 3). This decrease demonstrates an inhibitory activity which is able to overcome the beneficial effects of FCS on human chondrocytes.

### EXAMPLE III

### Production of Cartilage-Like Tissue Implant from Alginate Released Cell Slurry

The preparation of the cell slurry and the BCDM is performed as previously described in Examples I and II. Viscous BCDM is mixed with cell slurry in a ratio of 1 volume BCDM to 6 volumes cell slurry. The resultant suspension is placed in a silicon mould 2 mm thick and 20 mm in diameter. The base of the implant can be silicon (for chondral defect) or bone or bone substitute (for osteochondral defect). The implant is then overlaid with sterile dialysis tubing (MWCO 14,000), which is held securely in place by way of a metal frame. The implant is then placed in a 6 well plate and fed complete medium consisting of DMEM/F12, 50 μg/ml gentamicin, 10% human serum, 25 μg/ml ascorbic acid (Gibco Invitrogen, Basel, Switzerland), containing an effective amount of additional growth factors and/or chondroitin sulphate-4 and/or chondroitin sulphate-6. The cultures are kept at 37° C. in a humidified atmosphere of 5% CO2 in air with the medium replaced by fresh medium once every two days. After 7 to 14 days the dialysis membrane is removed and the implant cultured for a further period of time as necessary.

While there are shown and described presently preferred embodiments of the invention, it is to be distinctly understood that the invention is not limited thereto but may be otherwise variously embodied and practiced within the scope of the following claims.

What is claimed:

1. A method for the production of cartilage-like tissue in vitro comprising the following step:
cultivation of chondrogenic cells in presence of bivalent cation dependent constituents of the extracellular matrix (BCDM) and/or human serum wherein an inhibitory activity of cartilage maturation has been selectively removed from said human serum.

2. The method of claim 1, wherein said inhibitory activity is an inhibitory factor or inhibitory factors of cartilage growth and/or maturation.

3. The method of claim 2, wherein said inhibitory factor or factors have a molecular weight of less than 100,000 Dalton.

4. The method of claim 3, wherein said factor or factors have a molecular weight of less than 10,000 Dalton, preferably less than 8000 Dalton.

5. The method of claim 1, wherein said chondrogenic cells are cultivated in presence of a factor selected from the group consisting of chondroitin sulfate, interleukins, lipids, glucosamine, heparin sulphate, TGFβ, IGF-1, dexamethasone, FGF2 and PDGFββ.

6. The method of claim 1, wherein said chondrogenic cells are chondrocytes.

7. The method of claim 6, wherein said chondrocytes derive from chondrogenic cells which are seeded in a alginate gel and cultured for a time sufficient for
a) differentiation into chondrocytes and
b) biosynthesis and assembly of extracellular matrix.

8. The method of claim 6, wherein said chondrogenic cells are multiplied by anchorage dependent growth before seeded in the alginate gel.

9. The method of claim 1, wherein said chondrogenic cells stem from a tissue biopsy, from mesenchymal stem cells or stromal cells.

10. The method of claim 1, wherein said chondrogenic cells stem from a human being or animal.

11. The method of claim 1, wherein said chondrogenic cells are cultivated in a confined space.

12. The method of claim 11, wherein said chamber is at least in part made of a bone and/or bone substitute material, in particular hydroxyapatite or tricalcium phosphate.

13. The method of claim 11, wherein said BCDM are added to isolated chondrogenic cells before cultivation in a confined space.

14. The method of claim 1, wherein said bivalent cation dependent constituents of the extracellular matrix are isolated from a chondrocyte culture.

15. The method of claim 1, wherein the in vitro produced cartilage-like tissue or a compound formed of said cartilage-like tissue and bone or bone substitute is in a further step subjected to a mechanical treatment.

16. The method of claim 15, wherein during said mechanical treatment forces which have parallel and perpendicular components with respect to a surface of said cartilage-like tissue or said compound formed of said cartilage-like tissue and bone or bone substitute are applied.

17. The method of claim 15, wherein said cartilage-like tissue or the compound formed of said cartilage-like tissue and bone or bone substitute is subjected to mechanical stimulation by intermittent compression/deformation cycles.

18. An in vitro cartilage-like tissue or a compound formed of said cartilage-like tissue and bone or bone substitute obtained by the method of claim 1.

19. An implant for repairing an chondral and/or osteochondral defect comprising an in vitro cartilage-like tissue or the compound formed of said cartilage-like tissue and bone or bone substitute of claim 18.

20. A method for the treatment of a chondral and/or osteochondral defect in a subject wherein an in vitro cartilage-like tissue of claim 18 and/or an implant of claim 19 is applied to/implanted into said chondral and/or osteochondral defect.

21. The method of claim 20, wherein said subject is a human being or animal.

22. The method of claim 20, wherein said in vitro cartilage-like tissue is autologous tissue.

23. A method of joining several tissue patches for forming a substantially closed tissue surface comprising the steps of:
a) providing tissue patches of substantially polygonal shape, in particular tissue patches comprising cartilage-like tissue obtained by the method of claim 1 , and
b) arranging said polygonal tissue patches side by side in such a manner that they form a substantially closed tissue surface.

24. The method of claim 23, wherein the tissue patches used are part of tissue/bone composites.

25. The method of claim 23 or 24, wherein the tissue patches comprise a curved circumferential line.

26. A substantially closed tissue surface obtained by the method of claim 23.

27. An implant comprising the tissue surface of claim 26.

28. The implant of claim 27, wherein said implant is a cartilage-like/bone composite.

29. The implant of claim 27, wherein said implant comprises interlocking edges.

30. The method of claim 2 wherein the inhibitory factor is preferably a factor or factors inhibiting the production of cartilage matrix.

31. The method of claim 3 wherein the factor or factors have a molecular weight of less than 75,000 Dalton.

32. The method of claim 3 wherein the factor or factors have a molecular weight of less than 50,000 Dalton.

33. The method of claim 3 wherein the factor or factors have a molecular weight of less than 25,000 Dalton.

34. The method of claim 5 wherein the factor is IGF-1 long R3.

35. The method of claim 11 wherein the confined space is preferably a chamber whose confinements are in part porous or semipermeable.

36. The method of claim 13 wherein BCDM are added after multiplication of said chondrogenic cells.

37. The method of claim 14 wherein the chondrocyte culture is an alginate chondrocyte culture.
